(19) United States
(12) Patent Application Publication
(10) Pub. No.: US 2006/0008505 A1
(43) Pub. Date: Jan. 12, 2006

(54) Product

(76) Inventor: Gerard J. Brandon, Dun Laoghaire (IE)

Correspondence Address: JACOBSON HOLMAN PLLC, 400 SEVENTH STREET N.W., SUITE 600, WASHINGTON, DC, 20004, US

(21) Appl. No.: 11/176,580

(22) Filed: Jul. 8, 2005

Related U.S. Application Data
(60) Provisional application No. 60/585,838, filed on Jul. 8, 2004.

(51) Int. Cl. A61K 31/716; A61F 2/00

(52) U.S. Cl. 424/426; 514/57

(57) ABSTRACT

A delivery system for a haemostatic material comprises a self adhesive strip of a bioadhesive, especially pectin and a glycerol plasticiser. The haemostatic material may be a microdispersed oxidised cellulose which is dispersed in the strip. The strip is applied to control localised bleeding from surface skin wounds such as shaving nicks or minor cuts. The pectin dissolves in the blood, releasing the haemostatic material to control the bleeding.

The invention relates to a delivery system for delivery of a product such as a product to control bleeding, especially from surface skin wounds such as shaving nicks, or minor cuts.

### STATEMENTS OF INVENTION

According to the invention there is provided a delivery system comprising:
a strip of a carrier that is at least partially soluble in blood; and
a haemostatic material that is released as the material of the strip dissolves.

In one embodiment the carrier comprises a bioadhesive.

In another embodiment the bioadhesive is self adhesive.

In a further embodiment the carrier comprises pectin.

In one embodiment the carrier comprises a plasticiser.

In another embodiment the plasticiser comprises glycerine.

In a further embodiment the haemostatic material is a cellulose based material.

In one embodiment the cellulose material is oxidised cellulose.

In another embodiment the haemostatic material is microdispersed oxidised cellulose.

In a further embodiment the carrier comprises pectin in an amount of at least 30% by weight of the system. In one arrangement the pectin is present in an amount of at least 40% by weight of the system. In another arrangement the pectin is present in an amount of at least 50% by weight of the system. In a further arrangement the pectin is present in an amount of approximately 58% by weight of the system.

In one embodiment the glycerine is present in an amount of at least 20% by weight of the system. In another embodiment the glycerine is present in an amount of at least 25% by weight of the system. In a further embodiment the glycerine is present in an amount of approximately 30% by weight of the system.

In one embodiment the microdispersed oxidised cellulose is present in an amount of at least 5% by weight of the system. In another embodiment the microdispersed oxidised cellulose is present in an amount of from 10 to 15% by weight of the system.

In one embodiment the microdispersed oxidised cellulose is present in an amount of from 24 to 30% by weight of the system.

In another embodiment the system comprises a preservative such as potassium sorbate.

In a further embodiment the strip has a thickness of less than 200 microns, preferably less than 100 microns. In one arrangement the strip has a thickness of from 58 to 70 microns.

### DETAILED DESCRIPTION

The invention will be more clearly understood from the following description thereof, given by way of example only.

### EXAMPLE

The following formulation was prepared. All ingredients are in parts by weight.

| | |
|---|---|
| Microdispersed Oxidised Cellulose | 24 to 30% |
| (from Alltracel Pharma Limited) | |
| Pectin | 58% max |
| (from CP Kelco-food grade Genu type D slow set Z) | |
| Glycerine | 30% max |
| (any suitable food grade such as 99.5% grade | |
| from Natural Chemicals Ltd) | |
| Potassium Sorbate | 0.4% max |
| (Food Grade) | |

The microdispersed oxidised cellulose is a calcium/sodium salt of a random copolymer of anhydroglucouse (β-D-glucopyranose) and anhydroglucuronic (β-D-glucopyranic) acid. The product is prepared as described in WO98/33822A, the entire contents of which are herein incorporated by reference. The amount of oxidised cellulose released is about 20 to 30 g/m² which simulates the average amount of the material which may be applied as a dusting powder onto a wound area. The microdispersed oxidised cellulose is characterised by a high specific area and in contact with blood or tissue fluids readily forms a gel-like mat.

The microdispersed oxidised cellulose permits an easy and uniform incorporation into a carrier film. Because of the localised delivery it may be possible to achieve the same results with lower loading of microdispersed oxidised cellulose such as 10 to 15%.

Microdispersed oxidised cellulose is the preferred haemostatic material in view of its purity, stability and compatibility with the carrier however other haemostatic materials (which may be derived from cellulose) may be used alternatively or additionally.

The microdispersed oxidised cellulose material is intimately blended with the pectin, glycerol and potassium sorbate, and filtered. A thin film of the mixture is continuously applied to and spread onto a release paper and then air-dried. The product is then wound into a reel and subsequently the desired strips are cut to single strips about 20×30 mm, weight 50 to 60 mg and a thickness of 58 to 70 microns. A number of individual strips are then packed in a box.

The strips are intended for use as a means to help control minor bleeding from surface skin wounds such as shaving nicks, or minor cuts. They are intended for sale over-the-counter to both laymen patients and carers.

In use, the wound area is cleaned. An individual strip is removed from the box, applied directly over the wound area, and held in place for a few seconds. The portion of the strip in contact with the blood dissolves, releasing the microdispersed oxidised cellulose. When bleeding has stopped any remaining part of the strip maybe peeled off.

Pectin is the preferred carrier because it is a polysaccharide of plant origin with no known safety risks. There may be potential risks associated with animal based alternatives such as gelatine.

It is anticipated that plasticisers other than glycerine may be used. Similarly a preservative other that potassium sorbate may be used.

The invention is not limited to the embodiments herein before described which may be varied in detail.

1. A delivery system comprising:
a strip of a carrier that is at least partially soluble in blood; and
a haemostatic material that is released as the material of the strip dissolves.

2. A system as claimed in claim 1 wherein the carrier comprises a bioadhesive.

3. A system as claimed in claim 2 wherein the bioadhesive is self adhesive.

4. A system as claimed in claim 1 wherein the carrier comprises pectin.

5. A system as claimed in claim 1 wherein the carrier comprises a plasticiser.

6. A system as claimed in claim 5 wherein the plasticiser comprises glycerine.

7. A system as claimed in claim 1 wherein the haemostatic material is a cellulose based material.

8. A system as claimed in claim 7 wherein the cellulose material is oxidised cellulose.

9. A system as claim in claim 1 wherein the haemostatic material is microdispersed oxidised cellulose.

10. A system as claim in claim 1 wherein the carrier comprises pectin in an amount of at least 30% by weight of the system.

11. A system as claimed in claim 10 wherein the pectin is present in an amount of at least 40% by weight of the system.

12. A system as claimed in claim 10 wherein the pectin is present in an amount of at least 50% by weight of the system.

13. A system as claimed in claim 10 wherein the pectin is present in an amount of approximately 58% by weight of the system.

14. A system as claimed in claim 6 wherein the glycerine is present in an amount of at least 20% by weight of the system.

15. A system as claimed in claim 14 wherein the glycerine is present in an amount of at least 25% by weight of the system.

16. A system as claimed in claim 14 wherein the glycerine is present in an amount of approximately 30% by weight of the system.

17. A system as claimed in claim 9 wherein the microdispersed oxidised cellulose is present in an amount of at least 5% by weight of the system.

18. A system as claimed in claim 17 wherein the microdispersed oxidised cellulose is present in an amount of from 10 to 15% by weight of the system.

19. A system as claimed in claim 17 wherein the microdispersed oxidised cellulose is present in an amount of from 24 to 30% by weight of the system.

20. A system as claimed in claim 1 comprising a preservative such as potassium sorbate.

21. A system as claimed in claim 1 wherein the strip has a thickness of less than 100 microns.

22. A system as claimed in claim 1 wherein the strip has a thickness of from 58 to 70 microns.
